(19) United States
(12) Patent Application Publication
(10) Pub. No.: US 2023/0386678 A1
(43) Pub. Date: Nov. 30, 2023

(54) COMPUTER SYSTEM FOR CONTRAINDICATION IDENTIFICATION USING A DATA MODEL AND ELECTRONIC RECORDS

(71) Applicant: CERNER INNOVATION, INC., North Kansas City, MO (US)

(72) Inventors: Praveen Bhat GURPUR, Bengaluru (IN); Shaz Ul MP, Bengaluru (IN)

(21) Appl. No.: 17/824,440

(22) Filed: May 25, 2022

(51) Int. Cl. G16H 50/70; G06N 5/02; G16H 70/40; G16H 10/60

(52) U.S. Cl. CPC G16H 50/70; G06N 5/022; G16H 70/40; G16H 10/60

(57) ABSTRACT

Methods, systems, and computer-readable media are disclosed herein for generating, training, and deploying a specialized data model that is accessible through a computer software application for clinical decision support. Through the application and data model, a particular set of vaccine formulations for treating or preventing a condition are identified, the data model having a deep knowledge of the contraindications of each vaccine formulation. Using an individual-specific electronic record, data values are extracted by the application and used by the data model to determine whether a conflict is identifiable between the data values and the complex and varied contraindications of each vaccine formulation. The application preferentially presents conflict-free formulations as recommendation for administration to an individual that corresponds to the individual-specific electronic record and omits/prevents the display for those formulations having a conflict in a graphical user interface.

### TECHNICAL FIELD

The present disclosure generally relates to a machine-learning/artificial intelligence data model for data mining and evaluation of specialized electronic records.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

In brief and at a high level, this disclosure describes, among other things, methods, systems, and computer-readable media for an application a data model trained using web-based content mining and machine learning techniques to identify and/or predict complex vaccine contraindications relative to patient-specific electronic records.

A computerized method is provided in one aspect. The computerized method comprises running an application that integrates a data model trained with content extracted from a plurality of websites corresponding to a plurality of vaccine formulations associated with a plurality of conditions. In accordance with the method, an indication of a particular condition and an identifier are received via the application. Then, the identifier is matched to a particular electronic medical record (EMR) via the application and the particular condition is matched to a set of vaccine formulations that are specific to the particular condition via the data model. The set of vaccine formulations include two or more of the plurality of vaccine formulations that are specific to the particular condition. For each vaccine formulation in the set of vaccine formulations, a plurality of contraindications are identified via the data model. It is further determined, via the application, whether one or more of an allergy field, a medication field, or a status field in the particular EMR lacks a value. The application provides to the data model an indication for the data model to pause processing until the one or more of the allergy field, the medication field, or the status field receives an input of the value. Responsive to the input of the value to the one or more of the allergy field, the medication field, or the status field in the particular EMR, the application provides to the data model an indication to resume processing. For one or more of the vaccine formulations in the set, a conflict is identified by the data model between one or more of the plurality of contraindications and one or more of the allergy field, the medication field, and/or the status field in the particular EMR. For each of the one or more of the vaccine formulations for which the conflict is identified, it is determined to prevent presentation of an identifier of the vaccine formulation in a graphical user interface of the application. For one or more remaining vaccine formulations for which no conflict is identified, it is determined to present an identifier of the remaining vaccine formulation in the graphical user interface of the application. A graphical user interface is generated and displayed that includes the one or more remaining vaccine formulations for which no conflict is identified. Electronic documentation of an administration of at least one of the one or more remaining vaccine formulations to a patient is received, and the indication of administration of the at least one of the one or more remaining vaccine formulations is stored to the particular EMR.

Another aspect provides computer-executable instructions embodied thereon that, when executed via one or more processors, perform a method. Via one or more processors, the method includes ingesting, by a data model, content from a plurality of websites retrieved by the data model using URLs, the plurality of websites corresponding to a plurality of vaccine formulations associated with a plurality of conditions. Further, the method includes mapping, by the data model, a plurality of contraindications to each of the plurality of vaccine formulations. An indication of a particular condition and a patient identifier are received via an application that integrates the data model. The patient identifier is matched to a particular EMR that corresponds to one patient. Via the data model, the particular condition is matched to a set of vaccine formulations that are specific to the particular condition, the set of vaccine formulations including two or more of the plurality of vaccine formulations. For each vaccine formulation in the set of vaccine formulations, a plurality of contraindications are identified via the data model. Further, it is determined whether one or more of an allergy field, a medication field, or a status field in the particular EMR lacks a value. An indication is provided for the data model to pause processing until the one or more of the allergy field, the medication field, or the status field receives an input of the value. Responsive to the input of the value to the one or more of the allergy field, the medication field, or the status field in the particular EMR, an indication is provided to the data model to resume processing. For one or more of the vaccine formulations in the set of vaccine formulations, a conflict between one or more of the plurality of contraindications and the allergy field, the medication field, or the status field in the particular EMR is identified via the data model. For each of the one or more of the vaccine formulations for which the conflict is identified, it is determined to prevent presentation of an identifier of the vaccine formulation in a graphical user interface of the application. For one or more remaining vaccine formulations for which no conflict is identified, it is determined to present an identifier of the remaining vaccine formulation in the graphical user interface of the application. Then, the one or more remaining vaccine formulations for which no conflict is identified are presented in a graphical user interface of the application that is generated and caused to be displayed. In further aspects, an indication of administering at least one of the one or more remaining vaccine formulations to the one patient is received.

In another aspect, a system is provided. The system comprises one or more processors, a data model trained by ingesting content from a plurality of websites, and an application that uses the data model. Using the data model, and via the one or more processors, the application receives an indication of a particular condition and an identifier via the application. The application matches the identifier to a particular electronic record, and matches the particular condition to a set of vaccine formulations that are specific to the particular condition. For each vaccine formulation in the set of vaccine formulations, a plurality of contraindications are identified via the data model. For one or more of the vaccine formulations in the set of vaccine formulations, a conflict between one or more of the plurality of contraindications and the one or more fields is identified. The system then generates and causes presentation of a graphical user interface of the application, wherein the graphical user interface includes an identifier of each vaccine formulation in the set of vaccine formulations for which no conflict is identified and omits an identifier of each of the one or more vaccine formulations in the set for which the conflict is identified.

### BRIEF DESCRIPTION OF THE DRAWINGS

Aspects are described in detail below with reference to the attached drawings figures, wherein:

FIG. 1 is a block diagram of a system environment suitable to implement aspects of the present invention;

FIG. 2 is a block diagram of an application of the system of FIG. 1 in accordance with aspects described herein;

FIG. 3 is a flow diagram showing a method in accordance with aspects described herein;

FIG. 4 is a flow diagram showing another method in accordance with aspects described herein; and

FIG. 5 depicts an example computing environment in accordance with in accordance with aspects described herein.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

In aspects herein, a machine learning data model is created by training with specialized clinical information extracted in an autonomous manner from web-content, and the trained model can then be utilized through a computer application. That application intelligently scans a patient record for particular discrete data items that are extracted and utilized by the data model to evaluate a set of condition-specific vaccine formulations identified by the application. The data model and its intelligence, as well as patient records, are stored in one or more repositories. The data model's machine-learned intelligence can recognize which particular discrete data items create a conflict with particular contraindications of particular condition-specific vaccine formulations. The application omits or disregards those formulations for which the data model has identified a conflict when providing a recommendation that only includes those formulations that are conflict-free. The application can concurrently determine when particular discrete data items are missing or absent from the patient record, where these particular discrete data items are relevant to the conflict identification. The application can automatically prompt an end-user to obtain and input for those particular discrete data items (e.g., lab values, alphanumeric string). Based on receipt of the new information, the data model can automatically resume conflict processing using the new information in order to identify if there is a predicted or known conflict with particular contraindications of particular condition-specific vaccine formulations.

Aspects discussed hereinafter provide an improvement in the technical field of artificial intelligence deployment for utilization and incorporation in computer software that provides clinical decision support. Existing technology, at best, is limited to binary checks of only a few, non-complex parameters such as age or gender. Thus, existing technology is prone to errors of omission, meaning that such technology fails to recognize and fails to evaluate hundreds of parameters that cannot be evaluated in a binary manner. In contrast, aspects herein provide a data model that can automatically identify and/or predict conflicts between the complex information involving vaccine formulations and multiple factors/parameters stored in an electronic patient record well beyond age or gender, down to the examination of formulation-specific itemized ingredients. Aspects herein, particularly the trained data model and the application that provide a graphical user interface, improve over existing technology because the trained data model and application can access and evaluate data stored in intelligently-recognized specific fields of a patient-specific electronic health record (EHR) and/or patient-specific electronic medical record (EMR), and can make contraindication conflict determinations in real-time or near real-time based on the data model's knowledge. These specialized machine-learning aspects cannot be performed practically in the human, nor with a pen and paper, as the data model and application perform operations that are not, and cannot, be performed by humans.

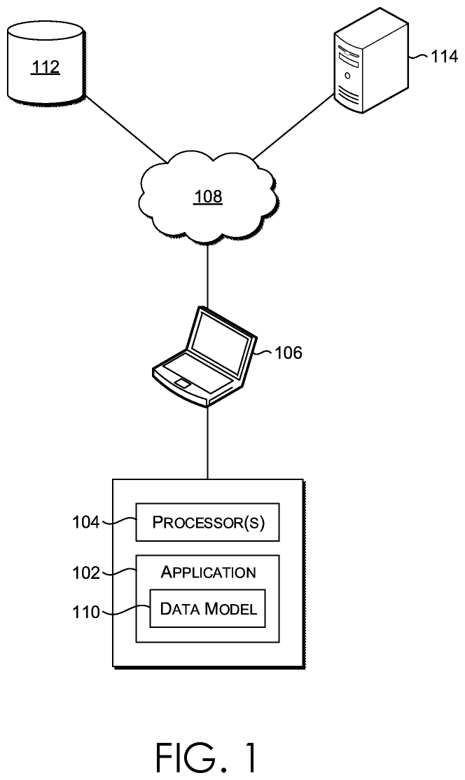

Beginning with FIG. 1, a system environment 100 is depicted in accordance with an aspect of the present invention. The system environment 100 includes an application 102, one or more processors 104, and an end-user computing device 106. The system environment 100 further includes a network 108, a data model 110, a repository 112 that is associated with the data model 110, and an electronic health information system 114 storing a plurality of electronic records.

The application 102 can perform complex computerized functions/tasks as further described hereinafter by utilizing the data model 110, as trained. In aspects, the complex computerized functions/tasks are invisible to the end-user of the end-user computing device 106 while output and outcomes are presented via one or more graphical user interfaces, such as recommendations, prompts for end user input, alerts, notices, or more.

The one or more processors 104 are hardware, such as an integrated electronic circuit that executes computer programming code that encodes instructions, stored in memory or other computer-readable media and/or computer-executable media, for example. The end-user computing device 106 can include the one or more processors 104 that executed computer-readable media and/or computer-executable media having programming instructions for running the application 102, either locally, remotely, and/or in a hybrid fashion (e.g., a portion of the application runs locally at the device while another portion runs remotely at a server). The end-user computing device 106 can be, for example, a laptop, a smartphone, a personal desktop-type computer (PC), a tablet, an electronic notebook, a smartwatch or other smart wearable device, a mobile computing device cart, a touchscreen device, or the like through which an end-user can access and interact with the application 102.

The network 108 may include one or more networks of short-range, mid-range, and/or long-range capabilities. Examples of the network 108 can include a Wide Area Network (WAN), a Local Area Network (LAN), a Wireless Local Area Network (WLAN), a cellular telecommunications network, a Wi-Fi network, a Wireless Metropolitan Area Network (WMAN), a Bluetooth® capable network, a fiber optic network, and any combination thereof. The network 108 may include local components and backhaul components, which are not shown for simplicity. The network 108 may include one or more interfaces that facilitate interoperability of electronic record and data exchanges between a plurality of local and/or remote systems that electronically communicate with the Electronic Health Information System (EHIS) 114, in some aspects.

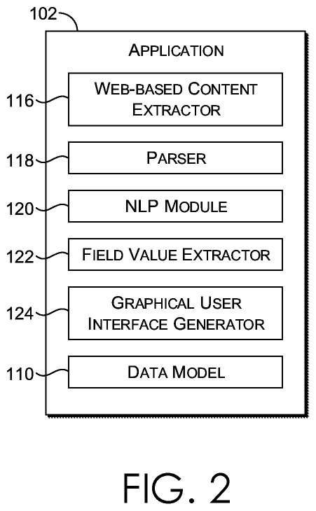

The data model 110 may be a flat-type data model, a hierarchal-type data model, a network-type data model, an entity-relationship-type data model, a dimensional data model, a relational data model, for example. In one example, the data model 110 is a multiclass decision tree model. In some aspects, the data model 110 leverages one or more of a web-based content extractor 116, a parser 118, a natural language processing module 120, a field value extractor 122, and/or a graphical user interface generator 124 associated with the application 102, as shown in FIG. 2.

The repository 112 is associated with the data model 110 in the example of FIG. 1. The repository can include physical memory, virtual memory, and/or cloud-based memory that stores the data model 110, training data, test data, and the like, for example. As such, the repository 112 may be any number of databases or data stores, such terms being used interchangeably herein. The repository 112 may store data and metadata in a specially structured manner or using a particular technological technique that is specific to the type of data model, for example, organized in records and sets, into tables, or into relational tables. In one aspect, the repository 112 may be a database management system (DBMS).

The Electronic Health Information System (EHIS) 114 is a specialized, secure, complex computing system that manages the storage, control, access to, editing of, and electronic transfer of plurality of electronic records across multiple interfaces and compatibility types. The EHIS 114 can include computerized administration systems, computerized laboratory systems, computerized pharmacy systems, and/or computerized coding and billing systems, for example. The EHIS may further manage patient data, clinical data, laboratory data, radiology data, pharmacy data, and more, for example, across one or more clinical entities such that the EHIS facilitates interoperability and electronic transport of such data in compliance with local and federal regulations. The EHIS 114 further stores a plurality of electronic records, such as electronic health records (EHRs) and/or electronic medical records (EMRs) for example. In some aspects, the EHIS, the application 102, and the data model 110 can utilize and recognize HealthLevel7 (HL7) message protocols, for example, as well clinical terminologies, classification systems, and/or coding, such as International Statistical Classification of Diseases and Related Health Problems (ICD-10), International Classification of Primary Care (ICPC), and/or systematized nomenclature of human and veterinary medicine (SNOMED).

In the system environment 100, the data model 110 is trained by ingesting content from a plurality of websites. In some aspects, the plurality of websites are called using one or more Uniform Resource Locators (URLs) to locate and load web-based content. In such aspects, discrete data items, metadata, images, text, and other web-based content are automatically extracted, parsed, recognized for context and/or meaning based on Natural Language Processing (NLP), and are ingested into the data model 110 as a training data set. Rendering the web-based content in a web browser is automated and/or optional. In one aspect, the plurality of websites correspond to a plurality of vaccine formulations that are associated with a plurality of conditions treatable using said vaccine formulations. In other aspects, when direct access to the structured database that stores the web-based content is accessible or available, the data model 110 can ingest the information directly from the database as a training data set. As used herein, a “condition” refers to a disease, virus, bacteria, parasite, and/or other pathological condition that is treatable and/or preventable through the administration of a specially developed vaccine that triggers an immune response sufficient to prevent death or serious illness that is otherwise caused by the condition. Examples of a condition include tetanus, poliovirus, pertussis, rotavirus, rubella, measles, mumps, pneumonia, tuberculosis, diphtheria, malaria, shingles, hepatitis B, human papillomavirus (HPV), severe acute respiratory syndrome coronavirus 1 (SARS-CoV-2, also referred to as COVID-19), and human immunodeficiency virus (HIV). Generally, a condition may be referred by the name of the pathogen (e.g., pertussis), a resulting disease (e.g., shingles results reactivated varicella-zoster virus), a location associated with the condition's discovery (e.g., Ebola virus), or an infected/discovering person. As used herein, a “vaccine formulation” refers to a particular mixture of compounds, chemical, and/or pharmacological molecules that is formulated to trigger an immune response that targets a particular condition or a combination of conditions, such that a formulation generally corresponds to a particular brand of a vaccine or particular manufacturer, each brand/manufacturer producing a proprietary or unique formulation relative to others. In some instances, a single vaccine formulation may treat a combination of conditions, such as a single vaccine formulation that treats all three conditions of Diphtheria, Tetanus, and Pertussis (DTaP). An example of vaccine formulations for DTaP include Daptacel® (manufactured by Sanofi®) and Infanrix® (manufactured by GlaxoSmithKline®). In some aspects, a single vaccine formulation may treat a single condition. One example of a single vaccine formulation treating a single condition is the vaccine formulation RTS,S/AS01 (referred to as Mosquirix®) that treats the condition of malaria. In one example, a single condition such as influenza may have many vaccine formulations: Aflura®, Fluad®, Fluarix®, Flublok®, FluLaval®, Fluzone®, and FluMist®. Each vaccine formulation for a particular condition or a particular combination of conditions, for example, may have the same, similar, or different routes of administration, approved ages for administration, ingredients, shelf-life, required or recommended sequence or quantity of doses, and/or characterizations (e.g., adjuvanted, inactivated pathogen/virus, recombinant, live pathogen/virus, live attenuated pathogen/virus, RNA/mRNA).

Accordingly, the data model 110 ingests the content of a plurality of websites that correspond to a plurality of vaccine formulations for one or more conditions. For example, one or more websites and webpages listing conditions and all of the vaccine formulations that are licensed for utilization in a designated country or region for said conditions can be ingested.

In such aspects, the data model 110 can identify contraindications using the web-based content. Examples of contraindications can include prior and/or current utilization of specific medications, prior and/or current receipt of specific therapeutic treatments, current pregnancy status, current patient age, prior and/or current history of particular disease/diagnosis (e.g., Thymic disorders), current immune system status (i.e., immunocompetence status), history of hypersensitivity and/or allergic reaction to ingredients, foods, and/or medications used in or to manufacture the vaccine formulation, prior history of allergic reaction to specific medications or therapies, and the like. As discussed herein, contraindications can include medications, medical histories, patient status, patient age, food allergies, medical allergies, and more, that are associated with a negative outcome such that administration of the vaccine formulation is not recommended. Contraindications can also include materials or mediums used in the manufacture of the vaccine formulation that may be associated with an allergy, e.g., a glass vial having a natural rubber latex stopper may be contraindicated for those with latex allergies; a vaccine formulation that contains an a component propagated using chicken embryos may be contraindicated for patients having allergies to egg proteins and/or feathers). Accordingly, the contraindications discussed herein are loosely categorized for ease of description and discussion but it will be understood that some of these non-limiting examples may easily fit into more than one category of contraindications.

The data model 110 can identify, for example, one or more medical or therapeutic treatments that are associated with a negative outcome from administration for one or more of the vaccine formulations, in aspects. Examples of medical treatments can include irradiation, antimetabolites, alkylating agents, cytotoxic drugs, corticosteroids, immunomodulatory drugs, and the like. Additionally, the data model 110 can identify, using the web-based content, one or more patient statuses that are associated with a negative outcome based on administration the vaccine formulation, in aspects. Examples of a patient status include immunocompromised, immunosuppressed, immunomodulation, active pregnancy, a first trimester of pregnancy, a second trimester of pregnancy, a third trimester of pregnancy, actively breastfeeding, an organ transplant recipient, a bone marrow transplant recipient, a current and/or prior diagnosis of cancer, an HIV positive status, and/or other status. Further, the data model 110 identifies a plurality of ingredients that are associated with each vaccine formulation, in aspects. Ingredients in the vaccine formulation can refer to therapeutically active substances/compounds and inactive substances/compounds (e.g., excipients, preservatives, stabilizers, fillers). Examples of ingredients can include deactivated virus/pathogen, live virus/pathogen, toxoids, vectors, polysaccharides, capsids, proteins, RNA, sorbitol, gelatin, nitrogen, and protamine sulfate.

The contraindications identified by the data model 110 can be optionally categorized as, for example, corresponding to one or more of an allergy, a medication, a status, and/or “other” contraindication by the data model 110 and/or the application 102. The term “allergy” and “hypersensitivity” are used interchangeably herein. Also, the terms “medication,” “drug,” “therapies,” and “treatment” are used interchangeably herein.

When identifying contraindications, the data model 110 can parse or scan the web content associated with each vaccine formulation to locate text blocks or other data organized into categories of under headers of “allergies,” “medical history,” and “medications” in the web-content. For example, the data model 110 may ingest and learn from text blocks associated with “medical history” to identify, extract, and store in association with one or more identifiers for that vaccine formulation, genetic conditions, non-communicable diseases, virus positivity (e.g., HIV, HBV, HCV), current or recent surgical procedures, specific medical diagnoses or events (e.g., heart attack, stroke, physical trauma), infectious diseases, and pregnancy status. In an example, the data model 110 ingests and learns from text defining contraindications such as “age” web-based content for the particular vaccine formulation. The data model 110 can extract data such as product information, package insert information, supporting documents, proper name, tradename, manufacturer, and indication data, for example, as shown in the webpages nested with the website: https://www.fda.gov/vaccines-blood-biologics/vaccines/vaccines-licensed-use-united-states

Based on the data model 110 ingesting content from the plurality of websites/webpages that correspond to the plurality of vaccine formulations associated with the plurality of conditions, the data model 110 maps specific contraindications to each of the plurality of vaccine formulations, and maps each vaccine formulation to a particular condition or combination of conditions. The data model 110 learns and stores the mappings of contraindications to particular vaccine formulations for particular conditions, as well as other associated data (e.g., identifiers, tradename) from the web-based content. In this manner, the data model 110 is trained using the webpage/website content as the training data set.

The data model 110, once trained, is utilized by the application 102. In some aspects, the application 102 can support of the data model 110. For example, the application 102 can include, integrate, and/or leverage one or more of a web-based content extractor 116, a parser 118, a natural language processing module 120, or any combination thereof, as shown in FIG. 2. In such aspects, the web-based content extractor 116 extracts data from the plurality of websites/webpages, a parser 118 parses the data extracted, and a natural language processing module 120 processes the data extracted. The web-based content extractor 116, the parser 118, and the natural language processing module 120 can provide the processed data to the data model 110 for ingestion and training. The web-based content extractor 116, the parser 118, and the natural language processing module 120 may be integrated into the application 102 and/or may be integrated with the data model 110, as shown in the example of FIG. 2. The application 102 can additionally or alternatively include a field value extractor 122 and/or a graphical user interface generator 124, as discussed herein.

The application 102 operates to receive an indication of a particular condition and an identifier, for example, based on user input, user selections, or other received data via a graphical user interface. For example, user input may specify a string of alphanumeric characters that identify one or more specific condition(s) such as “diphtheria and pertussis,” “polio,” “influenza,” or “flu.” The application 102 identifies, determines, and/or matches the particular condition to a set of vaccine formulations that are specific to the particular condition(s). For example, the application 102 can automatically connect to and query the repository 112 that is associated with the data model 110 using values and/or a string for an identifier that uniquely identifies the particular condition(s). In one example, the application 102 can query the repository 112 using one or more strings of “diphtheria,” “pertussis,” or “diphtheria and pertussis” based on the indication received. The application 102 can identify, based on the results of this query, that there are seven corresponding vaccine formulations recognized by the data model 110: Daptacel®, Infanrix®, Kinrix®, Pediarix®, Pentacel®, Quadracel®, and Vaxelis™.

For each vaccine formulation in the set of vaccine formulations that correspond to the condition(s) of the indication received, the application 102 identifies, determines, and/or matches each vaccine formulation in the set to a plurality of contraindications. The application 102 performs this identification and/or determination using the data model 110, which has deep knowledge of which plurality of contraindications correspond to which vaccine formulation(s), based on the training data set. The data model 110 provides the plurality of contraindications to the application 102 with specificity as to which contraindication(s) are associated with and/or linked to particular vaccine formulations for the condition(s) at issue.

The application 102 further identifies, determines, and/or matches the identifier received to a particular electronic record. For example, the application 102 can use the identifier to query the Electronic Health Information System (EHIS) 114 and locate one or more specific records, such as EHRs and/or EMRs that correspond to a particular patient. The application 102 may locate the particular electronic record using the identifier concurrently and/or simultaneously with the data model's identification and/or determination of the vaccine formulations that correspond to the condition(s). Alternatively, the application 102 may locate the particular electronic record using the identifier before or after the data model's identification and/or determination of the vaccine formulations that correspond to the condition(s). In either aspect, the application 102 locates the particular electronic record and the data model identifies the vaccine formulations in real-time or near real-time in response to the indication that was received. Examples of an identifier for locating an electronic record include a person's first and last name, a medical record number, a social security number, a date of birth, and the like, or any combination thereof.

In aspects, the application identifies and/or determines whether one or more fields in the particular electronic record have a value or string, and whether any fields lack a value or string, i.e., a field having a value of <null>, a field that does not include any alphanumeric characters or string, a field that has a value of “N/A” or is empty. As mentioned above, the application 102 can include a field value extractor 122 to perform this field value/string verification. The field value extractor 122 may identify and/or determine whether one or more fields in the particular electronic record lack a value. The field value extractor 122 can identify which particular field(s) in the electronic record is/are lacking information, for example. The field value extractor 122 can utilize natural language processing (NPL) techniques (and/or leverage the natural language processing module 120), one or more specialized computer-generated ontologies specific to the medical field and/or coding information (e.g., SNOMED) in determining whether the electronic record lacks some information. In some aspects, the field value extractor 122 scans the electronic record and particularly identifies one or more specific fields or a specific set of fields that correspond to allergies, medications, medical history, laboratory results, diagnoses, and the like. Once identified in the electronic record, the field value extractor 122 can determine whether these particular field(s) contain values and/or strings, for example, or lack values or strings. The field value extractor 122 extracts values and/or strings from each of the fields, and recognizes which values and/or strings are extracted from each particular field, when such fields are not empty. As such, the field value extractor 122 can extract the string “peanut” and the string “egg” from the allergies field in the electronic record, for example. The field value extractor 122 can extract “HIV positive” from another field in the electronic record, for example. The application 102 provides the values and/or strings extracted to the data model 110 in order to identify whether the values and/or strings correspond to, are associated with, are linked to, and/or match one or more of the contraindications for each of the vaccine formulations identified for the condition(s) indicated. Using these values and/or strings for particular fields, the data model 110 can perform conflict processing in order to automatically determine whether the values and/or strings are a full or partial match to one or more of the contraindications for each vaccine formulation, as further described herein.

When the application 102 and/or the field value extractor 122 determines that a field for one or more of allergy, medications, or medical history does not contain any values and/or strings (e.g., is empty of information), the application 102 provides an indication for the data model 110 to pause processing of the electronic record until the one or more fields receives an input of the value and/or string, in some aspects. The application 102 generates and communicates a prompt to the end-user computing device 106, wherein the prompt requests that an end-user provide one or more values and/or one or more strings for the particular field that is empty. For example, a prompt may communicate and request input for an empty “allergies” field in the electronic record, to which an input may be received of “none,” “n/a” (e.g., non-applicable), “unknown,” or “penicillin,” for example. It will be understood from this discussion however, that the automatic field value verification and prompt is an optional feature and may be utilized or triggered when an EHIS and/or EHRs/EMRs are accessible to the application 102. When an input is received for each of the one or more empty fields, the application 102 provides an indication to the data model to resume processing, as responsive to the input(s) of the value(s). As such, the input of values and/strings for each empty field are provided to the data model 110 for conflict processing of the newly-received or input values and/or strings relative to the contraindications for each vaccine formulation.

For one or more of the vaccine formulations in the set of vaccine formulations, the application 102 and/or data model 110 may identify a conflict between one or more of the plurality of contraindications for that vaccine formulation and the one or more fields in the electronic record. Based on scanning the fields in the electronic record for data that is stored or organized into particular headers or categories, such as “allergies” and “medical history,” the application 102 extracts the data in particular fields. The extracted data is used to query the repository 112, including contraindication(s) associated with each respective vaccine formulation in the set. The application 102 utilizes the data model 110 to query the repository 112 to locate the specifics for a particular vaccine formulation and to identify and evaluate contraindication data categorized as “Ingredients,” “Excipients,” and/or “Manufacturing Process Materials.” In aspects, the data model 110 can connect to and query other repositories to locate clinically standardized nomenclatures (e.g., ICD-10) which the data model 110 uses to match fields values/strings in the electronic record, such as a patient's diagnosis, to one or more of the plurality of contraindications of that vaccine formulation. In aspects, the application 102 and/or data model 110 can determine, identify, and/or match the contraindication(s) of a particular vaccine formulation to data in the particular electronic record using a code match, an exact value/string match, and/or a partial value/string match, for example. In this manner, the application 102 and/or data model 110 can query and analyze structured and unstructured data in performing the conflict processing. In some aspects, conflicts can be identified by the date model 110 using Named Entity Recognition (NER) techniques, String Matching (SM), computerized technologies, or any combination thereof. In one aspect, conflicts are identified using a combination of NER with SM. In that aspect, the information encoded or presented via HTML, Microsoft Word®, and/or PDF formatting in the websites are recognized independent of style, font, or type using NER. As such, NER recognizes that “Egg” “egg” and “EGG” all represent or refer to the same entity. In the same example, SM is utilized by the data model 110 to recognize “egg” within a patient-specific EHR/EMR. The data model 110 can use each distinct technique to recognize the information in different sources, and can then identify that “egg” within the patient-specific EHR/EMR is in conflict with “EGG” from the contraindications of the vaccine formulation obtained from the website, in such an example.

The application 102 and/or data model 110 can identify when there is conflict between the value in the allergy field in the particular electronic record and one or more ingredients of the vaccine formulation, the one or more ingredients corresponding to one or more of the plurality of contraindications. For example, the application 102 and/or data model 110 can identify that there is a conflict when the electronic record includes an allergy field value/string of “egg” and a particular vaccine formulation includes an ingredient value/string “egg,” which are recognized as a match, as linked, or as corresponding to each other based on the prior training of the data model 110. Additionally or alternatively, the application 102 and/or data model 110 can identify when there is conflict between the value in the medication field in the particular electronic record and one or more of the plurality of contraindications for the vaccine formulation. For example, application 102 and/or data model 110 can identify that there is a conflict when the electronic record includes a medication field value/string of “adalimumab” and a particular vaccine formulation includes a contraindication of value/string “immunosuppressed,” which are recognized as a match, as linked, or as corresponding to each other based on the prior training of the data model 110. Additionally or alternatively, the application 102 and/or data model 110 can identify when there is conflict between the status field in the particular electronic record and one or more of the plurality of contraindications for the vaccine formulation. For example, application 102 and/or data model 110 can identify that there is a conflict when the electronic record includes a status field value/string of “second trimester” and a particular vaccine formulation includes a contraindication of value/string “pregnancy,” which are recognized as a match, as linked, or as corresponding to each other based on the prior training of the data model 110. The application 102 and/or data model 110 perform conflict processing for one or more relevant fields (e.g., allergies, medications, medical history, gender, age, date of birth) in the electronic record against the vaccine formulation contraindications to identify all conflicts for each vaccine formulation in the set. Additionally, the application 102 and/or data model 110 can recognize that one or more relevant fields are temporally impacted. In other words, only the medications that are currently being utilized as listed in the electronic record are relevant to the conflict processing, while historical medications that are no longer being utilized or prescribed are not relevant to the conflict processing and can be disregarded. In such an example, the data model 110 may particularly compare the current form of values/strings in specific fields in the electronic record, such as surgical procedures, infectious disease/diagnosis, pregnancy status, and medications, while disregarding historical data in such fields. Other fields may always be examined as relevant to the conflict processing, such as genetic conditions, immunocompetence status, and non-communicable disease/diagnosis, for example.

For each of the one or more of the vaccine formulations for which the conflict is identified, the application 102 determines that it will prevent presentation of an identifier of the vaccine formulation in a graphical user interface of the application. In other words, if a vaccine formulation has one or more predicted and/or identified conflicts, that vaccine formulation is ruled out by the application 102 and the application 102 will not provide that vaccine formulation as a recommendation for administration. In one example, if a vaccine formulation has even one predicted and/or identified conflict, that vaccine formulation is ruled out by the application 102 and the application 102 will not provide that vaccine formulation as a recommendation for administration. Alternatively, formulations for which conflicts are identified may be displayed in a graphical user interface in some aspect so as to convey the conflict to an end-user.

In some aspects, when a vaccine formulation has even one predicted and/or identified conflict based on the data model, the data model may stop evaluating that vaccine formulation, and devote processing resources to the other remaining vaccine formulations for which a conflict has not been identified at that time in processing.

For one or more remaining vaccine formulations for which no conflict is identified and/or predicted, the application 102 determines that it will present an identifier of each of the one or more remaining vaccine formulations in the graphical user interface of the application. The application 102 and/or data model 110 performs this conflict processing for each condition being evaluated in the present instance, albeit it will be understood that the conflict processing for different or for multiple conditions can be conducted concurrently or sequentially in various aspects.

As stated above, the application 102 can include a graphical user interface generator 124. The graphical user interface generator 124 generates and causes presentation of a graphical user interface of the application, wherein the graphical user interface includes an identifier of each remaining vaccine formulation in the set for which no conflict is identified and omits an identifier of each of the one or more vaccine formulations in the set for which the conflict is identified. The remaining vaccine formulation(s) for which no conflict is identified are displayed as recommendation(s) for administration to an individual that corresponds to the electronic record that was matched and processed. The sequence or order in which the one or more remaining vaccine formulations are displayed can be an alphabetized list, a ranked list, and/or a randomized order, in various aspects. The one or more remaining vaccine formulations may be presented, for example, as a ranked list based on greatest levels of local stock levels/availability, lowest cost to a patient, fewest side-effects, or other factors of that remaining vaccine formulation relative to the other remaining formulations. The graphical user interface generator 124 omits any identifier for the vaccine formulations having a conflict, in some aspects. Alternatively, in some aspects, the graphical user interface generator 124 may include an identifier for the vaccine formulations having a conflict, but these formulations may be displayed as an alert to prevent their administration to an individual, where the identifier may be displayed using red-colored text or other visual depiction for caution and avoidance (e.g., “!” or “DO NOT ADMINISTER FluMist®”).

In some aspects, the application 102 and/or data model 110 may utilized a probability of impact (PI) formula to resolve a scenario where an individual is determined to have at least one conflict with each vaccine formulation. For example, the data model 110 may determine that there are two conflicts for the individual, based on the electronic record, with a first vaccine formulation, but only one conflict for the individual, based on the electronic record, with a second vaccine formulation. The data model 110 may calculate a probability of impact for each of the conflicts to determine which of the first or second vaccine formulation may be associated with a lower or low contraindication risk relative to each other. When the probability of impact score is be determined to be less than 0.5, for example, the application 102 may provide the first and/or second vaccine formulations as recommendations alongside additional warnings that detail the specific conflicts identified in the graphical user interface of the application 102. Alternatively, both the first and second vaccinations may be presented with corresponding warnings that detail the specific conflicts identified, as well as the respective probability impact score of each in the graphical user interface of the application 102.

Having described the system environment 100 and components thereof, it will be understood by those of ordinary skill in the art that system environment 100 is but one example of a suitable system and is not intended to limit the scope of use or functionality of the present invention. Similarly, system environment 100 should not be interpreted as imputing any dependency and/or any requirements with regard to each component and combination(s) of components illustrated in FIGS. 1 and 2. It will be appreciated by those of ordinary skill in the art that the locations of components illustrated in FIGS. 1 and 2 are an example, as other methods, hardware, software, components, and devices for establishing a communication links between the components shown in FIGS. 1 and 2, may be utilized in implementations of the present invention. It will be understood to those of ordinary skill in the art that the components may be connected in various manners, hardwired or wireless, and may use intermediary components that have been omitted or not included in FIGS. 1 and 2 for simplicity's sake. As such, the absence of components from FIGS. 1 and 2 should be not be interpreted as limiting the present invention to exclude additional components and combination(s) of components. Moreover, though components are represented in FIGS. 1 and 2 as singular components, it will be appreciated that some aspects may include a plurality of devices and/or components such that FIGS. 1 and 2 should not be considered as limiting the number of a device or component.

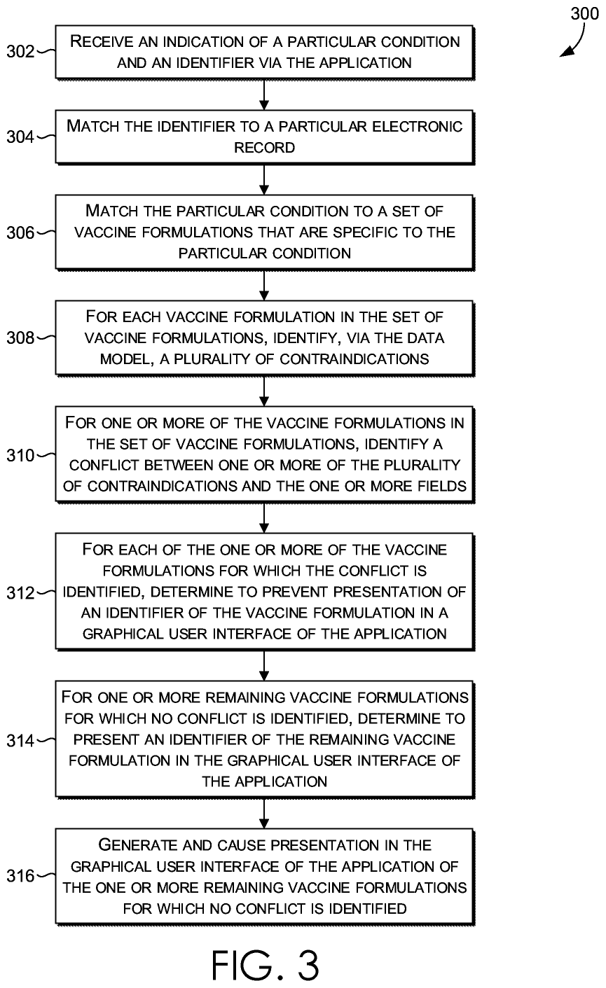

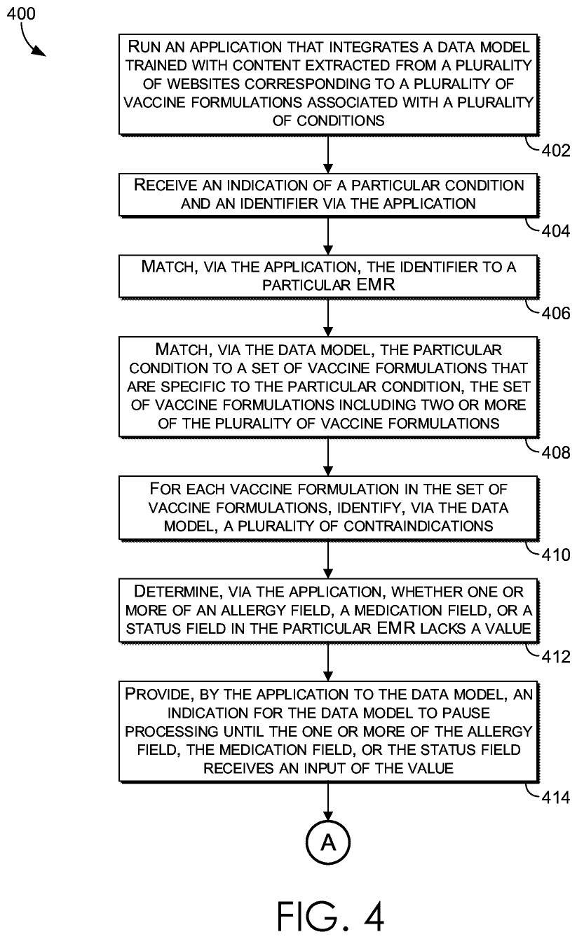

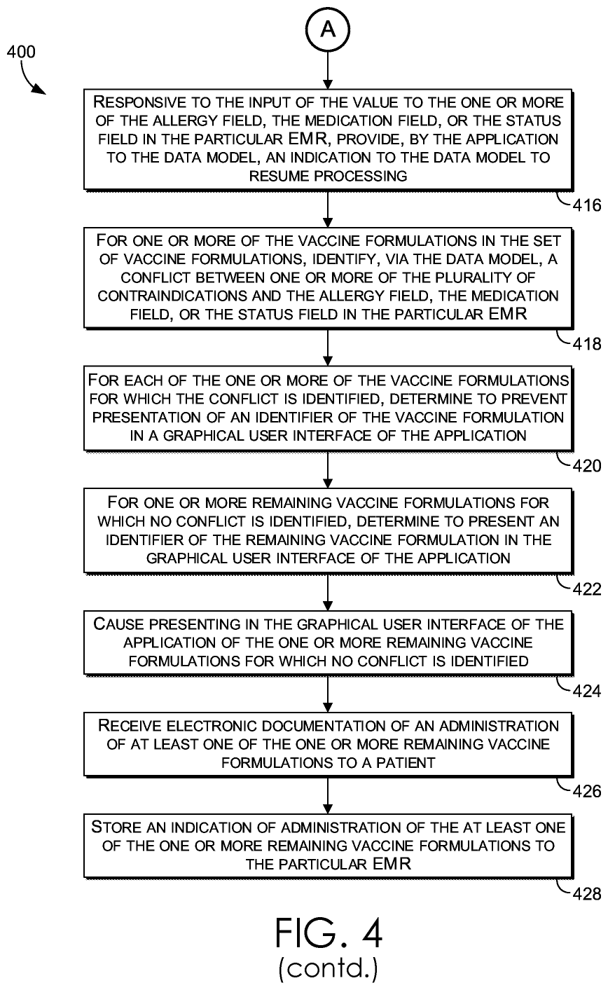

Turning now to FIGS. 3 and 4, methods are provided. As discussed below, the methods can be computer-implemented and/or performed using software, hardware, component(s), and/or device(s) depicted in the example of FIGS. 1 and 2. In one aspect, one or more non-transitory computer-readable storage media having computer-readable instructions or computer-readable program code portions embodied thereon, for execution via one or more processors, can be used to implement and/or perform the methods. For example, computer-readable instructions or computer-readable program code portions can specify the performance of the methods, can specify a sequence of steps of the methods and/or can identify particular component(s) of software and/or hardware for performing one or more of the steps of the methods, in aspects. The computer-readable instructions or computer-readable program code portions can correspond to an application and/or an application programming interface (API), in some aspects. In one aspect, the application or API can implement and/or perform the methods.

In FIG. 3, the method 300 includes receiving an indication of a particular condition and an identifier via an application at block 302, such as the application 102 of FIGS. 1 and 2. The identifier is matched to a particular electronic record, shown at block 304. In some aspects, the application 102 matches the identifier to particular electronic record, such a patient-specific EHR and/or EMR that is stored in the EHIS 114. Further, the particular condition is matched to a set of vaccine formulations that are specific to the particular condition, shown at block 306. In some aspects, the data model 110 matches the condition of the indication to a set of vaccine formulations that are specific to the particular condition. In various aspects, only a single vaccine formulation may be identified as corresponding to the particular condition(s) indicated in some situations, but the present methods are discussed using examples with multiple formulations for demonstration. As such, in some instances, the set of vaccine formulations can include two or more distinct formulations that correspond to the same condition or combination of condition(s). At block 308, for each vaccine formulation in the set of vaccine formulations, the data model identifies a plurality of contraindications. For one or more of the vaccine formulations in the set of vaccine formulations, a conflict is identified between one or more of the plurality of contraindications and one or more fields in the electronic record, shown at block 310. For example, a value or string in an allergy field may be matched to a particular contraindication of a specific vaccine formulation in the set. At block 312, for each of the one or more of the vaccine formulations for which the conflict is identified, it is determined by the application that an identifier of the vaccine formulation is to be prevented from being displayed in a graphical user interface of the application, e.g., will not be displayed. For one or more remaining vaccine formulations for which no conflict is identified, it is determined that an identifier of the remaining vaccine formulation(s) are to be presented in the graphical user interface of the application, shown at block 314. Then, a graphical user interface of the application is generated and the identifier(s) of the one or more remaining vaccine formulations for which no conflict is identified, shown at block 316. The conflict-free vaccine formulations are presented as recommendations in the graphical user interface for administration to an individual that is associated with the electronic record that has been evaluated.

In FIG. 4, another method 400 is provided. As discussed previously, the method 400 may be performed using a data model that has been trained. In such examples, the data model can be trained by ingesting content that is automatically extracted from the plurality of websites corresponding to the plurality of vaccine formulations associated with the plurality of conditions. In such aspects, the data model identifies a plurality of ingredients associated with the vaccine formulation, when identifying contraindications. Additionally or alternatively, in such aspects, the data model identifies one or more medical treatments that are associated with a negative outcome from the administration the vaccine formulation, when identifying contraindications. Additionally or alternatively, the data model identifies one or more patient statuses that are associated with a negative outcome based on the administration the vaccine formulation, when identifying contraindications. The data model can further map each of the plurality of contraindications to each of the plurality of vaccine formulations to which they correspond, such information and intelligence being stored in a repository for utilization by the application in real-time or near real-time evaluations. Once trained, the data model can accurately support the conflict determinations and recommendations of the application. The data model can also automatically be retrained, for example, based on a user input, periodically, and/or based on a triggering event.

Beginning at block 402, an application is run that integrates a data model trained with content extracted from a plurality of websites corresponding to a plurality of vaccine formulations associated with a plurality of conditions. At block 404, an indication of a particular condition and an identifier are received via the application. At block 406, the identifier is matched to a particular EMR, via the application. At block 408, the particular condition is matched, via the data model, to a set of vaccine formulations that are specific to the particular condition, the set of vaccine formulations including two or more of the plurality of vaccine formulations. At block 410, a plurality of contraindications are identified via the data model for each vaccine formulation in the set of vaccine formulations. In various aspects, each vaccine formulation may have one or more contraindications.

Then, at block 412, via the application, it is determined whether one or more of an allergy field, a medication field, or a status field in the particular EMR lacks a value. At block 414, the application provides an indication for the data model to pause processing until the one or more of the allergy field, the medication field, or the status field receives an input of the value. At block 416, responsive to the input of the value to the one or more of the allergy field, the medication field, or the status field in the particular EMR, the application provides an indication to the data model to resume processing (e.g., of conflict identification and prediction for each formulation in the set against the patient-specific EMR).

At block 418, for one or more of the vaccine formulations in the set of vaccine formulations, a conflict between one or more of the plurality of contraindications and the allergy field, the medication field, or the status field in the particular EMR is identified via the data model. In some instances, a conflict is identified between the value in the allergy field in the particular EMR and one or more ingredients of the vaccine formulation, the one or more ingredients corresponding to one or more of the plurality of contraindications. In another instance, a conflict is identified between the value in the medication field in the particular EMR and one or more of the plurality of contraindications for the vaccine formulation. In yet another instance, a conflict is identified between the status field in the particular EMR and one or more of the plurality of contraindications for the vaccine formulation. It will be understood that any combination of such conflicts can be identified. In some aspects, once any conflict is identified for a particular vaccine formulation, processing of the remaining fields in the electronic record against the contraindication of the particular vaccine formulation may be stopped and disregarded as that particular vaccine formulation will not be recommended, as further discussed below. Such selective determinations to stop processing some formulations, as made by the data model and/or application, can be used to conserve memory and processing resources, and to speed up the delivery of recommendations.

At block 420, for each of the one or more of the vaccine formulations for which the conflict is identified, the application determines that presentation of an identifier of the vaccine formulation is to be prevented in a graphical user interface of the application. It will be understood that in some instances, depending on the EMR information and the contraindications of the vaccine formulation set, there may be no conflicts identified. Continuing to block 422, for one or more remaining vaccine formulations for which no conflict is identified, the application determines that an identifier of the remaining vaccine formulation is to be presented in the graphical user interface of the application. At block 424, the application generates and causes presentation of the identifier for the one or more remaining vaccine formulations for which no conflict is identified in the graphical user interface of the application. At block 426, electronic documentation of an administration of at least one of the one or more remaining vaccine formulations to a patient is received, in further aspects. At block 428, an indication of the administration of the at least one of the remaining vaccine formulations is stored to the particular EMR, in such further aspects. For example, an indication of administration of the at least one of the one or more remaining vaccine formulations to the one patient can be stored in the particular EMR, wherein the application is authorized to securely and electronically communicate with an electronic health records system.

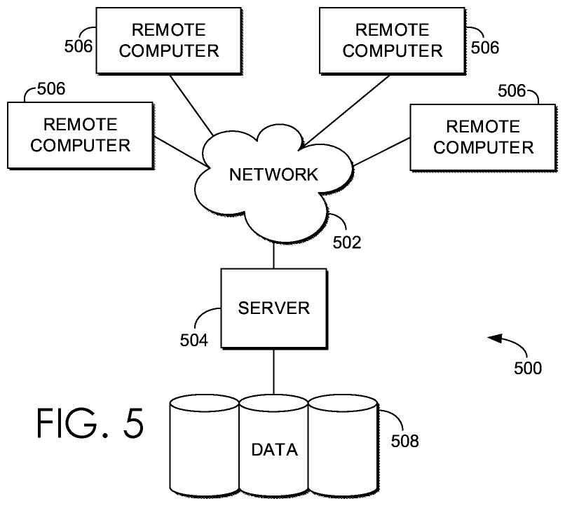

Turning now to with FIG. 5, an example of an computing environment 500 is depicted, in accordance with an aspect of the present invention. It will be understood by those of ordinary skill in the art that the computing environment 500 is just one example of a suitable computing environment and is not intended to limit the scope of use or functionality of the present invention. Similarly, the computing environment 500 should not be interpreted as imputing any dependency and/or any requirements with regard to each component and combination(s) of components illustrated in FIG. 5. It will be appreciated by those having ordinary skill in the art that the connections illustrated in FIG. 5 are also examples as other methods, hardware, software, and devices for establishing a communications link between the components, devices, systems, and entities, as shown in FIG. 5, may be utilized in implementation of the present invention. Although the connections are depicted using one or more solid lines, it will be understood by those having ordinary skill in the art that the example connections of FIG. 5 may be hardwired or wireless, and may use intermediary components that have been omitted or not included in FIG. 5 for simplicity's sake. As such, the absence of components from FIG. 5 should be not be interpreted as limiting the present invention to exclude additional components and combination(s) of components. Moreover, though devices and components are represented in FIG. 5 as singular devices and components, it will be appreciated that some aspects may include a plurality of the devices and components such that FIG. 5 should not be considered as limiting the number of a device or component.

Continuing, the computing environment 500 of FIG. 5 is illustrated as being a distributed environment where components and devices may be remote from one another and may perform separate tasks. The components and devices may communicate with one another and may be linked to each other using a network 502. The network 502 may include wireless and/or physical (e.g., hardwired) connections. Examples of networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 502, generally, provides the components and devices access to the Internet and web-based applications.

The computing environment 500 comprises a computing device 504, which may be in the form of a server. Although illustrated as one component in FIG. 5, the present invention may utilize a plurality of local servers and/or remote servers in the computing environment 500. The computing device 504 may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA®) local bus, and Peripheral Component Interconnect (PCI) bus, also known as Mezzanine bus.

The computing device 504 may include or may have access to computer-readable media. Computer-readable media can be any available media that may be accessed by computing device 504, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the computing device 504. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In aspects, the computing device 504 uses logical connections to communicate with one or more remote computers 506 within the computing environment 500. In aspects where the network 502 includes a wireless network, the computing device 504 may employ a modem to establish communications with the Internet, the computing device 504 may connect to the Internet using Wi-Fi or wireless access points, or the server may use a wireless network adapter to access the Internet. The computing device 504 engages in two-way communication with any or all of the components and devices illustrated in FIG. 5, using the network 502. Accordingly, the computing device 504 may send data to and receive data from the remote computers 506 over the network 502.

Although illustrated as a single device, the remote computers 506 may include multiple computing devices. In an aspect having a distributed network, the remote computers 506 may be located at one or more different geographic locations. In an aspect where the remote computers 506 is a plurality of computing devices, each of the plurality of computing devices may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some aspects, the remote computers 506 is physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; veterinarians; students; and the like. In other aspects, the remote computers 506 may be physically located in a non-medical setting, such as a packing and shipping facility or deployed within a fleet of delivery or courier vehicles.

Continuing, the computing environment 500 includes a data store 508. Although shown as a single component, the data store 508 may be implemented using multiple data stores that are communicatively coupled to one another, independent of the geographic or physical location of a memory device. Examples of data stores may store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. Examples of data stores may also store data in the form of electronic records, for example, electronic medical records of patients, transaction records, billing records, task and workflow records, chronological event records, and the like.

Generally, the data store 508 includes physical memory that is configured to store information encoded in data. For example, the data store 508 may provide storage for computer-readable instructions, computer-executable instructions, data structures, data arrays, computer programs, applications, and other data that supports the functions and action to be undertaken using the computing environment 500 and components shown in example of FIG. 5.

In a computing environment having distributed components that are communicatively coupled via the network 502, program modules may be located in local and/or remote computer storage media including, for example only, memory storage devices. Aspects of the present invention may be described in the context of computer-executable instructions, such as program modules, being executed by a computing device. Program modules may include, but are not limited to, routines, programs, objects, components, and data structures that perform particular tasks or implement particular abstract data types. In aspects, the computing device 504 may access, retrieve, communicate, receive, and update information stored in the data store 508, including program modules. Accordingly, the computing device 504 may execute, using a processor, computer instructions stored in the data store 508 in order to perform aspects described herein.

Although internal components of the devices in FIG. 5, such as the computing device 504, are not illustrated, those of ordinary skill in the art will appreciate that internal components and their interconnection are present in the devices of FIG. 5. Accordingly, additional details concerning the internal construction device are not further disclosed herein.

Regarding FIGS. 1 through 6, it will be understood by those of ordinary skill in the art that the environment(s), system(s), and/or methods(s) depicted are not intended to limit the scope of use or functionality of the present aspects. Similarly, the environment(s), system(s), and/or methods(s) should not be interpreted as imputing any dependency and/or any requirements with regard to each component, each step, and combination(s) of components or step(s) illustrated therein. It will be appreciated by those having ordinary skill in the art that the connections illustrated the figures are contemplated to potentially include methods, hardware, software, and/or other devices for establishing a communications link between the components, devices, systems, and/or entities, as may be utilized in implementation of the present aspects. As such, the absence of component(s) and/or steps(s) from the figures should be not be interpreted as limiting the present aspects to exclude additional component(s) and/or combination(s) of components. Moreover, though devices and components in the figures may be represented as singular devices and/or components, it will be appreciated that some aspects can include a plurality of devices and/or components such that the figures should not be considered as limiting the number of a devices and/or components.

It is noted that aspects of the present invention described herein with reference to block diagrams and flowchart illustrations. However, it should be understood that each block of the block diagrams and/or flowchart illustrations can be implemented in the form of a computer program product, an entirely hardware embodiment, a combination of hardware and computer program products, and/or apparatus, systems, computing devices/entities, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code can be performed sequentially such that one instruction is retrieved, loaded, and executed at a time. In some aspects, retrieval, loading, and/or execution can be performed in parallel such that multiple instructions are retrieved, loaded, and/or executed together. Thus, such aspects can produce specifically-configured machines performing the steps or operations specified in the block diagrams and flowchart illustrations. Accordingly, the block diagrams and flowchart illustrations support various combinations of aspects for performing the specified instructions, operations, or steps.

Additionally, as should be appreciated, various aspects of the present disclosure described herein can also be implemented as methods, apparatus, systems, computing devices/entities, computing entities, and/or the like. As such, aspects of the present disclosure can take the form of an apparatus, system, computing device, computing entity, and/or the like executing instructions stored on a computer-readable storage medium to perform certain steps or operations. However, aspects of the present disclosure can also take the form of an entirely hardware aspect performing certain steps or operations.

Many different arrangements of the various components depicted, as well as components not shown, are possible without departing from the scope of the claims below. Aspects of our technology have been described with the intent to be illustrative rather than restrictive. Alternative aspects will become apparent readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

What is claimed is:

1. A system comprising:
one or more processors;
a data model trained by ingesting content from a plurality of websites; and
an application that uses the data model, and via the one or more processors:
receives an indication of a particular condition and an identifier via the application;
matches the identifier to a particular electronic record;
matches the particular condition to a set of vaccine formulations that are specific to the particular condition;
for each vaccine formulation in the set of vaccine formulations, identifies, via the data model, a plurality of contraindications;
for one or more of the vaccine formulations in the set of vaccine formulations, identifies a conflict between one or more of the plurality of contraindications and the one or more fields; and
generates and causes presentation of a graphical user interface of the application, wherein the graphical user interface includes an identifier of each vaccine formulation in the set of vaccine formulations for which no conflict is identified and omits an identifier of each of the one or more vaccine formulations in the set for which the conflict is identified.

2. The system of claim 1, wherein the plurality of websites correspond to a plurality of vaccine formulations associated with a plurality of conditions, and wherein the one or more fields correspond to at least one of an allergy field, a medication field, or a status field.

3. The system of claim 2, wherein the application further:
identifies the conflict between the value in the allergy field in the particular electronic record and one or more ingredients of the vaccine formulation, the one or more ingredients corresponding to one or more of the plurality of contraindications.

4. The system of claim 2, wherein the application further:
identifies the conflict between the value in the medication field in the particular electronic record and one or more of the plurality of contraindications for the vaccine formulation.

5. The system of claim 2, wherein the application further:
identifies the conflict between the status field in the particular electronic record and one or more of the plurality of contraindications for the vaccine formulation.

6. The system of claim 2, wherein the application further:
identifies, via the data model, a plurality of ingredients associated with the vaccine formulation.

7. The system of claim 2, wherein the application further:
identifies, via the data model, one or more medical treatments that are associated with a negative outcome from administration the vaccine formulation.

8. The system of claim 2, wherein the application further:
identifies, via the data model, one or more patient statuses that are associated with a negative outcome based on administration the vaccine formulation.

9. The system of claim 1, wherein the application further:
determines whether one or more fields in the particular electronic record lack a value;
provides an indication for the data model to pause processing until the one or more fields receives an input of the value; and
responsive to the input of the value to the one or more fields, provides an indication to the data model to resume processing;

10. A computerized method comprising:
running an application that integrates a data model trained with content extracted from a plurality of websites corresponding to a plurality of vaccine formulations associated with a plurality of conditions;
receiving an indication of a particular condition and an identifier via the application;
matching, via the application, the identifier to a particular EMR;
matching, via the data model, the particular condition to a set of vaccine formulations that are specific to the particular condition, the set of vaccine formulations including two or more of the plurality of vaccine formulations;
for each vaccine formulation in the set of vaccine formulations, identifying, via the data model, a plurality of contraindications;
determining, via the application, whether one or more of an allergy field, a medication field, or a status field in the particular EMR lacks a value;
providing, by the application to the data model, an indication for the data model to pause processing until the one or more of the allergy field, the medication field, or the status field receives an input of the value;
responsive to the input of the value to the one or more of the allergy field, the medication field, or the status field in the particular EMR, providing, by the application to the data model, an indication to the data model to resume processing;
for one or more of the vaccine formulations in the set of vaccine formulations, identifying, via the data model, a conflict between one or more of the plurality of contraindications and the allergy field, the medication field, or the status field in the particular EMR;
for each of the one or more of the vaccine formulations for which the conflict is identified, determining to prevent presentation of an identifier of the vaccine formulation in a graphical user interface of the application;
for one or more remaining vaccine formulations for which no conflict is identified, determining to present an identifier of the remaining vaccine formulation in the graphical user interface of the application;
causing presenting of the identifiers of the one or more remaining vaccine formulations for which no conflict is identified in the graphical user interface of the application;
receiving electronic documentation of an administration of at least one of the one or more remaining vaccine formulations to a patient; and
storing an indication of administration of the at least one of the one or more remaining vaccine formulations to the particular EMR.

11. The method of claim 10, further comprising training the data model with the content automatically extracted from the plurality of websites corresponding to the plurality of vaccine formulations associated with the plurality of conditions.

12. The method of claim 11, wherein for one or more of the vaccine formulations in the set of vaccine formulations, identifying, by the data model, the conflict between the one or more of the plurality of contraindications and the allergy field, the medication field, or the status field in the particular EMR further comprises:
identifying the conflict between the value in the allergy field in the particular EMR and one or more ingredients of the vaccine formulation, the one or more ingredients corresponding to one or more of the plurality of contraindications.

13. The method of claim 11, wherein for one or more of the vaccine formulations in the set of vaccine formulations, identifying, by the data model, the conflict between the one or more of the plurality of contraindications and the allergy field, the medication field, or the status field in the particular EMR further comprises:
identifying the conflict between the value in the medication field in the particular EMR and one or more of the plurality of contraindications for the vaccine formulation.

14. The method of claim 11, wherein for one or more of the vaccine formulations in the set of vaccine formulations, identifying, by the data model, the conflict between the one or more of the plurality of contraindications and the allergy field, the medication field, or the status field in the particular EMR further comprises:
identifying the conflict between the status field in the particular EMR and one or more of the plurality of contraindications for the vaccine formulation.

15. The method of claim 11, wherein for each vaccine formulation in the set of vaccine formulations, identifying, by the data model, the plurality of contraindications comprises:
identifying, by the data model, a plurality of ingredients associated with the vaccine formulation.

16. The method of claim 11, wherein for each vaccine formulation in the set of vaccine formulations, identifying, by the data model, the plurality of contraindications comprises:
identifying, by the data model, one or more medical treatments that are associated with a negative outcome from the administration the vaccine formulation.

17. The method of claim 11, wherein for each vaccine formulation in the set of vaccine formulations, identifying, by the data model, the plurality of contraindications comprises:
identifying, by the data model, one or more patient statuses that are associated with a negative outcome based on the administration the vaccine formulation.

18. One more non-transitory computer-readable media having computer-executable instructions embodied thereon that, when executed via one or more processors, perform a method, the media comprising:
via one or more processors;
ingesting, by a data model, content from a plurality of websites retrieved by the data model using URLs, the plurality of websites corresponding to a plurality of vaccine formulations associated with a plurality of conditions;
mapping, by the data model, a plurality of contraindications to each of the plurality of vaccine formulations;
receiving an indication of a particular condition and a patient identifier via an application that integrates the data model;
matching the patient identifier to a particular EMR that corresponds to one patient;
matching, via the data model, the particular condition to a set of vaccine formulations that are specific to the particular condition, the set of vaccine formulations including two or more of the plurality of vaccine formulations;
for each vaccine formulation in the set of vaccine formulations, identifying, via the data model, the plurality of contraindications;
determining whether one or more of an allergy field, a medication field, or a status field in the particular EMR lacks a value;
providing an indication for the data model to pause processing until the one or more of the allergy field, the medication field, or the status field receives an input of the value;
responsive to the input of the value to the one or more of the allergy field, the medication field, or the status field in the particular EMR, providing an indication to the data model to resume processing;
for one or more of the vaccine formulations in the set of vaccine formulations, identifying, via the data model, a conflict between one or more of the plurality of contraindications and the allergy field, the medication field, or the status field in the particular EMR;
for each of the one or more of the vaccine formulations for which the conflict is identified, determining to prevent presentation of an identifier of the vaccine formulation in a graphical user interface of the application;
for one or more remaining vaccine formulations for which no conflict is identified, determining to present an identifier of the remaining vaccine formulation in the graphical user interface of the application;
generating and causing presentation of the identifiers of the one or more remaining vaccine formulations for which no conflict is identified in the graphical user interface of the application;
receiving an indication of administering at least one of the one or more remaining vaccine formulations to the one patient.

19. The media of claim 18, further comprising identifying, via the data model, a plurality of ingredients associated with the vaccine formulation.

20. The media of claim 19, wherein for one or more of the vaccine formulations in the set of vaccine formulations, identifying, by the data model, the conflict between the one or more of the plurality of contraindications and the allergy field, the medication field, or the status field in the particular EMR further comprises one or more of:
identifying the conflict between the value in the allergy field in the particular EMR and one or more ingredients of the vaccine formulation, the one or more ingredients corresponding to one or more of the plurality of contraindications;
identifying the conflict between the value in the medication field in the particular EMR and one or more of the plurality of contraindications for the vaccine formulation; or
identifying the conflict between the status field in the particular EMR and one or more of the plurality of contraindications for the vaccine formulation.
